(19) United States
(12) Patent Application Publication
(10) Pub. No.: US 2020/0113506 A1
(43) Pub. Date: Apr. 16, 2020

(54) HEALTH MONITORING METHOD, WEARABLE DEVICE, HEALTH MONITORING SYSTEM, AND COMPUTER READABLE STORAGE MEDIUM

(71) Applicant: BOE TECHNOLOGY GROUP CO., LTD., Beijing (CN)

(72) Inventors: Changjun LIU, Beijing (CN); Tianxiao ZHAO, Beijing (CN); Zhichao LI, Beijing (CN); Tianyue ZHAO, Beijing (CN)

(21) Appl. No.: 16/411,074

(22) Filed: May 13, 2019

(30) Foreign Application Priority Data
Oct. 11, 2018 (CN) 201811184629.0

(51) Int. Cl. A61B 5/00; G16H 50/20; G16H 40/67; G16H 10/65; G16H 80/00; A61B 5/0205

(52) U.S. Cl. CPC A61B 5/4343; G16H 50/20; G16H 40/67; G16H 10/65; G16H 80/00; A61B 5/02411; A61B 5/6802; A61B 5/4362; A61B 5/742; A61B 5/02055; A61B 5/0022

(57) ABSTRACT

The present application provides a health monitoring method, a wearable device, a health monitoring system, and a computer readable storage medium. The health monitoring method includes: acquiring pregnant woman health reference information; transmitting the pregnant woman health reference information and pregnant woman identity information in the wearable device to a remote medical server terminal; and receiving pregnant woman health diagnosis information from the remote medical server terminal, wherein the pregnant woman health diagnosis information is determined according to the pregnant woman health reference information and the pregnant woman identity information.

### CROSS-REFERENCE TO RELATED APPLICATION(S)

The present application claims priority to the Chinese Patent Application 201811184629.0, filed on Oct. 11, 2018, which is incorporated herein by reference in its entirety.

### TECHNICAL FIELD

The present disclosure relates to the field of information processing technology, and more particularly, to a health monitoring method, a wearable device, a health monitoring system, and a computer readable storage medium.

### BACKGROUND

With the economic development and social progress, people's requirements for quality of life are getting higher and higher, people's health awareness is becoming stronger, and people's requirements for daily health care and health monitoring are becoming more and more prominent, especially for health monitoring of pregnant women.

Currently, for the health monitoring required for pregnant women, it is necessary to go to a medical institution (for example, a hospital) for inspection regularly, which takes more time and requires more labor costs, and is less convenient. Therefore, how to improve the convenience of health monitoring of pregnant women is a problem required to be solved.

### SUMMARY

The present disclosure provides a health monitoring method, a wearable device, a health monitoring system, and a computer readable storage medium.

According to a first aspect of the embodiments of the present disclosure, there is provided a health monitoring method. The health monitoring method comprises:

acquiring pregnant woman health reference information;

transmitting the pregnant woman health reference information and pregnant woman identity information in the wearable device to a remote medical server terminal; and

receiving pregnant woman health diagnosis information from the remote medical server terminal, wherein the pregnant woman health diagnosis information is determined according to the pregnant woman health reference information and the pregnant woman identity information.

In some embodiments, the pregnant woman health reference information and the pregnant woman identity information in the wearable device are transmitted through a cloud server, and the pregnant woman health diagnosis information is received through the cloud server.

In some embodiments, the health monitoring method further comprises:

transmitting a pregnancy start time in the wearable device to a cloud server; and

receiving, from the cloud server, a fetal development pattern map at a current pregnancy time determined based on the pregnancy start time.

In some embodiments, the health monitoring method further comprises:

transmitting the pregnant woman health reference information and the pregnant woman identity information to a storage device for storage.

In some embodiments, the health monitoring method further comprises:

transmitting the pregnant woman health reference information and the pregnant woman identity information to a cloud server, so that the cloud server determines pregnant woman health reference information belonging to a same pregnant woman according to the pregnant woman identity information, and establishes a pregnant woman personal health file.

In some embodiments, the health monitoring method further comprises:

displaying at least one of the pregnant woman health reference information, the pregnant woman identity information, or the pregnant woman health diagnosis information.

According to a second aspect of the embodiments of the present disclosure, there is provided a wearable device for health monitoring. The wearable device comprises:

an acquisition unit configured to acquire pregnant woman health reference information;

a communication unit configured to communicate with a remote medical server terminal; and

a processor, configured to:

control the acquisition unit to acquire the pregnant woman health reference information;

control the communication unit to transmit the pregnant woman health reference information and pregnant woman identity information in the wearable device to the remote medical server terminal; and

control the communication unit to receive pregnant woman health diagnosis information from the remote medical server terminal, wherein the pregnant woman health diagnosis information is determined according to the pregnant woman health reference information and the pregnant woman identity information.

In some embodiments, the pregnant woman health reference information and the pregnant woman identity information in the wearable device are transmitted through a cloud server, and the pregnant woman health diagnosis information is received through the cloud server.

In some embodiments, the processor is further configured to:

control the communication unit to transmit a pregnancy start time in the wearable device to a cloud server; and

control the communication unit to receive, from the cloud server, a fetal development pattern map at a current pregnancy time determined based on the pregnancy start time.

In some embodiments, the processor is further configured to:

control the communication unit to transmit the pregnant woman health reference information and the pregnant woman identity information to a storage device for storage.

In some embodiments, the processor is further configured to:

control the communication unit to transmit the pregnant woman health reference information and the pregnant woman identity information to a cloud server, so that the cloud server determines pregnant woman health reference information belonging to a same pregnant woman according to the pregnant woman identity information, and establishes a pregnant woman personal health file.

In some embodiments, the wearable device further comprises a display unit, configured to display at least one of the pregnant woman health reference information, the pregnant woman identity information, or the pregnant woman health diagnosis information.

According to a third aspect of the embodiments of the present disclosure, there is provided a computer readable storage medium, having stored thereon executable instructions which, when executed by a processor, implement the steps of the method according to any of the embodiments described above.

According to a fourth aspect of the embodiments of the present disclosure, there is provided a health monitoring system, comprising:

the wearable device according to any of the embodiments described above; and

a remote medical server terminal in communication with the communication unit in the wearable device, and configured to determine the pregnant woman health diagnosis information according to the pregnant woman health reference information and the pregnant woman identity information received from the wearable device, and transmit the pregnant woman health diagnosis information to the wearable device.

In some embodiments, the health monitoring system further comprises:

a storage device, configured to store the pregnant woman health reference information and the pregnant woman identity information; and

a cloud server configured to determine pregnant woman health reference information belonging to a same pregnant woman according to the pregnant woman identity information, and establish a pregnant woman personal health file.

In some embodiments, the cloud server is further configured to receive a pregnancy start time in the wearable device, determine a current pregnancy time based on the pregnancy start time, construct a fetal development pattern map at the current pregnancy time, and transmit the fetal development pattern map to the wearable device.

It should be understood that the above general description and the following detailed description are intended to be only exemplary and illustrative, and cannot limit the present disclosure.

### BRIEF DESCRIPTION OF THE DRAWINGS

The accompanying drawings here, which are incorporated in the specification and constitute a part of the specification, illustrate embodiments according to the present disclosure, and are used to explain the principles of the present disclosure together with the specification.

FIG. 1 is a flowchart illustrating an operation of a health monitoring method according to an embodiment of the present disclosure;

FIG. 2 is a flowchart illustrating an operation of a health monitoring method according to another embodiment of the present disclosure;

FIG. 3 is a flowchart illustrating an operation of a health monitoring method according to yet another embodiment of the present disclosure;

FIG. 4 is a block diagram of a wearable device according to an embodiment of the present disclosure;

FIG. 5 is a block diagram of a health monitoring system according to an embodiment of the present disclosure; and

FIG. 6 is a block diagram of a health monitoring system according to another embodiment of the present disclosure.

### DETAILED DESCRIPTION

Exemplary embodiments will be described in detail herein, wherein examples of the embodiments are illustrated in the accompanying drawings. When the accompanying drawings are referred to in the following description, the same numbers in different accompanying drawings represent the same or similar elements, unless otherwise indicated. The implementations described in the following exemplary embodiments do not represent all implementations according to the present disclosure. Instead, they are merely examples of apparatuses and methods according to some aspects of the present disclosure as detailed in the appended claims.

Currently, pregnant women need to go to medical institutions for health inspection. They need to spend a certain amount of transportation time and labor costs on their way to the medical institutions. It also takes a certain amount of time and labor costs to register for inspection and waits for inspection in the medical institutions. Therefore, it not only takes time and labor costs, but also is less convenient.

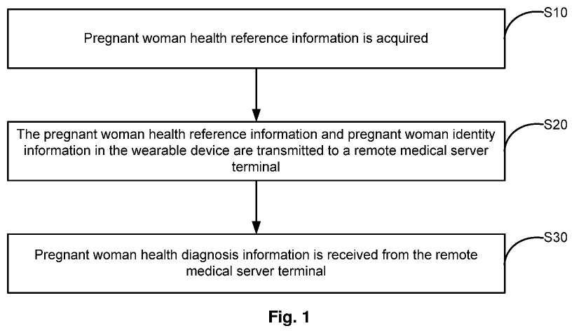

Accordingly, the embodiments of the present disclosure provide a health monitoring method, which is applied to a wearable device. As shown in FIG. 1, the method comprises the following steps.

In step S10, pregnant woman health reference information is acquired.

In step S20, the pregnant woman health reference information and pregnant woman identity information in the wearable device are transmitted to a remote medical server terminal.

In step S30, pregnant woman health diagnosis information is received from the remote medical server terminal, wherein the pregnant woman health diagnosis information is determined according to the pregnant woman health reference information and the pregnant woman identity information.

In some embodiments, the method may further comprise: displaying at least one of the pregnant woman health reference information, the pregnant woman identity information, or the pregnant woman health diagnostic information.

The wearable device is a portable device which may be worn directly on a person. The wearable device is more than just a hardware device, and may also implement more functions through software support, data interaction, cloud interaction etc. The wearable device is, for example, a smart watch, a smart bracelet, smart glasses etc.

The pregnant woman health reference information is related information characterizing physical conditions of a pregnant woman. Since the pregnant woman has a fetus in her body, the pregnant woman health reference information not only comprises physical condition information of the pregnant woman herself, but also comprises the related information of the fetus in the pregnant woman. The physical condition information of the pregnant woman herself comprises, for example, abdominal body surface temperature of the pregnant woman and a number of pulse beats of the pregnant woman etc., and the related information of the fetus in the pregnant woman comprises, for example, fetal heart information of the fetus, which comprises, for example, a number of heartbeats of the fetus and a heart rate of the fetus etc.

The pregnant woman health reference information may comprise at least one of fetal heart information of the fetus in the pregnant woman, the abdominal body surface temperature of the pregnant woman, and the number of pulse beats of the pregnant woman.

For the health conditions of the pregnant woman, the fetal heart information of the fetus in the pregnant woman may reflect conditions of the fetus in a uterus of the pregnant woman. When fetal hypoxia occurs for various reasons, the fetal heart may change very sensitively, and the heart rate in the fetal heart information is also an indicator reflecting whether a central nervous system of the fetus is developing normally.

It is conducive to discovering abnormal conditions of the fetus by monitoring the fetal heart information, so as to reduce the incidence of suffocation or even death of the fetus due to hypoxia.

The abdominal body surface temperature of the pregnant woman may reflect whether the body temperature of the pregnant woman is abnormal, and the number of pulse beats of the pregnant woman is conducive to timely detecting whether the pregnant woman is over-tired and whether the heart is abnormal etc.

The pregnant woman identity information represents related information of an identity of the pregnant woman, for example, a name, an age, a weight, a height, a blood type, and an identity number etc. of the pregnant woman.

The fetal heart information of the fetus in the pregnant woman, the abdominal body surface temperature of the pregnant woman and the number of pulse beats of the pregnant woman are important indicators reflecting the physical conditions of the pregnant woman herself and the development conditions of the fetus.

The pregnant woman identity information may be input by the pregnant woman through a user interface in the wearable device, and then stored by the wearable device; or may be transmitted to the wearable device through another device (for example, a terminal device, a mobile phone, a computer, etc. used by the pregnant woman) which has stored the pregnant woman identity information, and received and then stored by the wearable device.

The remote medical server terminal may be connected to the wearable device through a network, and may provide remote medical services to users, and the remote medical server terminal is, for example, a private doctor server terminal, a community hospital server terminal, a pregnant woman and child health care server terminal, and a central hospital server terminal etc.

The wearable device may transmit the pregnant woman health reference information and the pregnant woman identity information to the remote medical server terminal. The remote medical server terminal may diagnose the health conditions of the pregnant woman according to the health reference information and the pregnant woman identity information, and determine pregnant woman health diagnosis information. For example, the remote medical server terminal may determine whether a weight growth of the fetus in the pregnant woman is abnormal according to the weight of the pregnant woman, whether the pregnant woman has a fever through the abdominal body surface temperature of the pregnant woman, whether the heart rate of the pregnant woman is abnormal by comparing the number of pulse beats of the pregnant woman with a normal number of pulse beats, whether the fetal development is normal or the fetus is hypoxic according to the number of heartbeats of the fetus and the heart rate of the fetus etc.

The remote medical server terminal transmits the determined pregnant woman health diagnosis information to the wearable device for display, so that the pregnant woman may know the health conditions of herself in time, and if abnormality is discovered, the pregnant woman may go to a medical institution for further inspection and treatment in time, which avoids serious consequences due to late discovery of the abnormality, for example, suffocation or even death etc. of the fetus due to hypoxia.

It may be seen from the above description that the health monitoring method may be applied to the wearable device, and after the pregnant woman wears the wearable device, the pregnant woman may know the health conditions of herself and the fetus in her body in time according to the pregnant woman health reference information displayed by the wearable device, and may know professional health diagnosis information fed back by the remote medical server terminal in time, which may reduce a number of inspections in a medical institution, thereby reducing the time and labor costs, and improving the convenience and timeliness of monitoring the health of the pregnant woman.

In some embodiments, transmitting the health reference information and the stored pregnant woman identity information to the remote medical server terminal, as described in above step S20, comprises:

transmitting the pregnant woman health reference information and the stored pregnant woman identity information to the remote medical server terminal through a cloud server.

Receiving and displaying pregnant woman health diagnosis information determined by the remote medical server terminal according to the pregnant woman health reference information and the pregnant woman identity information, as described in the above step S30, comprises:

receiving and displaying the pregnant woman health diagnosis information determined by the remote medical server terminal according to the pregnant woman health reference information and the pregnant woman identity information through the cloud server.

In the present embodiment, the remote medical server terminal exchanges information with the wearable device through the cloud server, and both the remote medical server terminal and the wearable device are connected to the cloud server through a network. The wearable device transmits the pregnant woman health reference information and the pregnant woman identity information to the cloud server; after receiving the information, the cloud server transmits the information to the remote medical server terminal; the remote medical server terminal transmits the determined pregnant woman health diagnosis information to the cloud server; and the cloud server feeds back the received information to the wearable device.

The cloud server may usually be connected to various devices through a network, and the remote medical server terminal exchanges information with the wearable device through the cloud server without constructing a network through which the wearable device is connected to the remote medical server terminal, which reduces the cost.

The cloud server is, for example, a cloud platform or a network server etc.

Of course, the wearable device may be directly connected to the remote medical server terminal through a network. Thereby, the wearable device directly transmits the pregnant woman health reference information and the pregnant woman identity information to the remote medical server terminal, and the remote medical server terminal may also directly transmit the pregnant woman health diagnosis information to the wearable device.

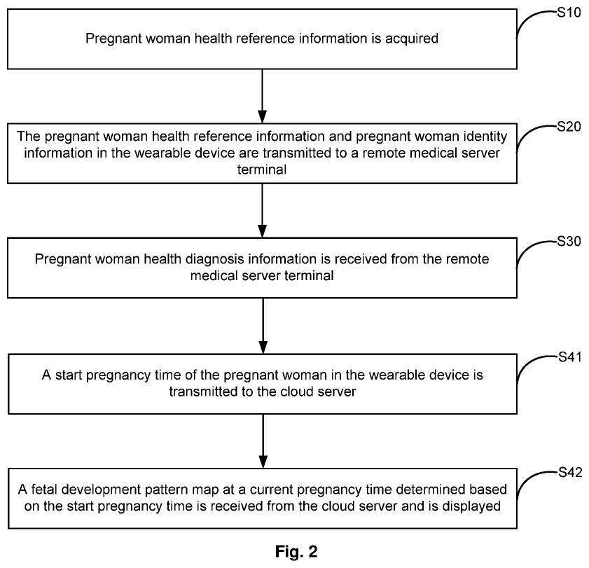

In some embodiments, as shown in FIG. 2, the method further comprises the following steps.

In step S41, a pregnancy start time of the pregnant woman in the wearable device is transmitted to the cloud server.

In step S42, a fetal development pattern map at a current pregnancy time determined based on the pregnancy start time is received from the cloud server and is displayed.

The pregnant woman may go through a long period of time from the beginning of pregnancy to childbirth (fetal birth), which may be referred to as a pregnancy cycle. A pregnancy start time of the pregnancy cycle is medically defined as a first day of a last menstrual period of the pregnant woman, and the pregnancy cycle is 280 days. However, since women have different menstrual cycles, the pregnancy cycle may normally have a deviation of 1 to 2 weeks.

The pregnancy start time may be stored by the wearable device after the pregnancy start time is input by the pregnant woman through the user interface of the wearable device, or is transmitted to the wearable device by another device which has stored the pregnancy start time, and is received and then stored by the wearable device.

Since the cloud server has strong information processing capability, in order to reduce the hardware cost of the wearable device, the wearable device itself does not determine the fetal development pattern map, but transmits the pregnancy start time to the cloud server, and the cloud server determines the fetal development pattern map at the current pregnancy time based on the pregnancy start time, and then the cloud server transmits the fetal development pattern map to the wearable device.

The cloud server may calculate the current pregnancy time according to the pregnancy start time and a current time, and the current pregnancy time is a period from the pregnancy start time to the current time, which may be accurate to a certain day of a year, or the current pregnancy time is in units of weeks, that is, gestational weeks.

During a process of fetal development in the pregnant woman, the fetus may have different characteristics and patterns, which may reflect the development of the fetus in various stages. The whole development process of the fetus may be divided into 40 gestational weeks from a first week to a 40th week in units of gestational weeks.

The fetal development pattern map at the current pregnancy time may be the fetal development pattern map in a gestational week in which the current pregnancy time is located. The fetal development pattern map may be displayed on the wearable device, so that the pregnant woman may more intuitively feel the development conditions of the fetus in her body at the current pregnancy time.

The above fetal development pattern map is a pattern map in ideal conditions of the fetus determined according to the current pregnancy time, and is not actually detected for the pregnant woman individually. The fetal development pattern map may be a two-dimensional map, a three-dimensional map or a four-dimensional map.

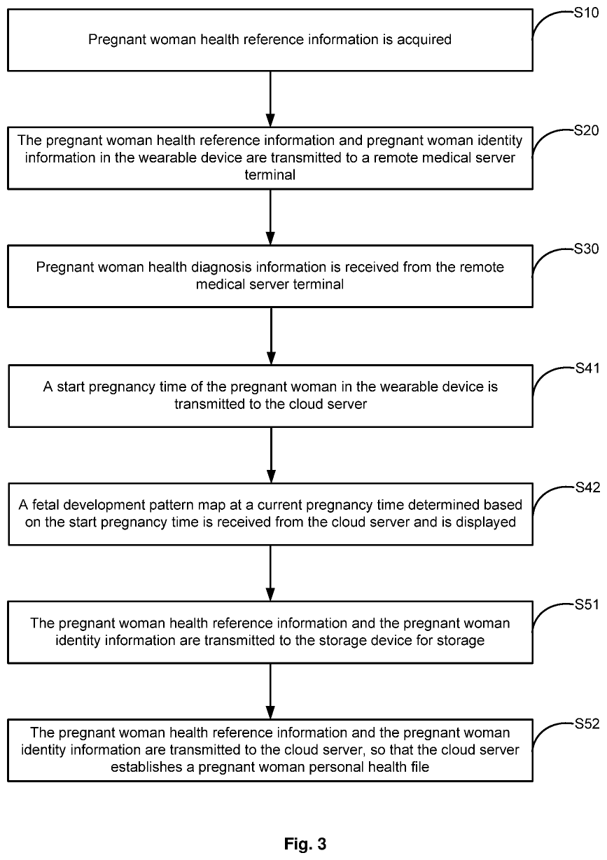

In some embodiments, as shown in FIG. 3, the method may further comprise the following steps.

In step S51, the pregnant woman health reference information and the pregnant woman identity information are transmitted to the storage device for storage.

In some embodiments, step S51 is performed based on a first voice instruction. The wearable device may have a voice interaction function, and the pregnant woman, as a user, may input an instruction in a voice form. The wearable device may analyze the voice instruction (referred to as the first voice instruction herein) to determine a command to be executed, and then transmit the pregnant woman health reference information and the pregnant woman identity information to the storage device, and the storage device stores the received pregnant woman health reference information and the pregnant woman identity information.

The first voice instruction described above is, for example, a pronunciation comprising “transmit” and “storage device” in a voice, and the wearable device may recognize the pronunciation of “transmit” and “storage device” from the first voice instruction, and then transmit the above information to the storage device.

The wearable device is controlled to transmit information in a voice manner, which is convenient to operate, and since the wearable device usually has a small data storage capacity, the pregnant woman health reference information and the pregnant woman identity information are stored by the storage device for reference and inquiry.

The above storage device may be a storage device used by the pregnant woman or a family comprising the pregnant woman, which is a device capable of storing information in various forms, for example, a mobile phone, a tablet computer, and a desktop computer etc.

Further, the method may further comprise the following steps.

In step S52, the pregnant woman health reference information and the pregnant woman identity information are transmitted to the cloud server, so that the cloud server determines pregnant woman health reference information belonging to the same pregnant woman according to the pregnant woman identity information, and establishes a pregnant woman personal health file.

In some embodiments, step S51 is performed based on a second voice instruction.

In the present embodiment, the wearable device may transmit the pregnant woman health reference information and the pregnant woman identity information received in real time to the cloud server, and the cloud server is a server or platform disposed on the network, and may receive a large amount of other data and information etc., comprising, for example, pregnant woman health reference information and pregnant woman identity information of a plurality of pregnant women. In order to process a variety of information, the cloud server may classify the health reference information according to the pregnant woman identity information, determine the pregnant woman health reference information belonging to the same pregnant woman, and then establish a pregnant woman personal health file for inquiry later.

The pregnant woman personal health file is a file created by the cloud server according to a large amount of pregnant woman health information which is received in real time, comprising pregnant woman health information which is detected in real time.

The second voice instruction described above is, for example, a pronunciation comprising “transmit” and “cloud server” in a voice, and the wearable device may recognize the pronunciation of “transmit” and “cloud server” from the second voice instruction, and then transmit the above information to the cloud server.

The cloud server may further transmit the established pregnant woman personal health file to the remote medical server terminal.

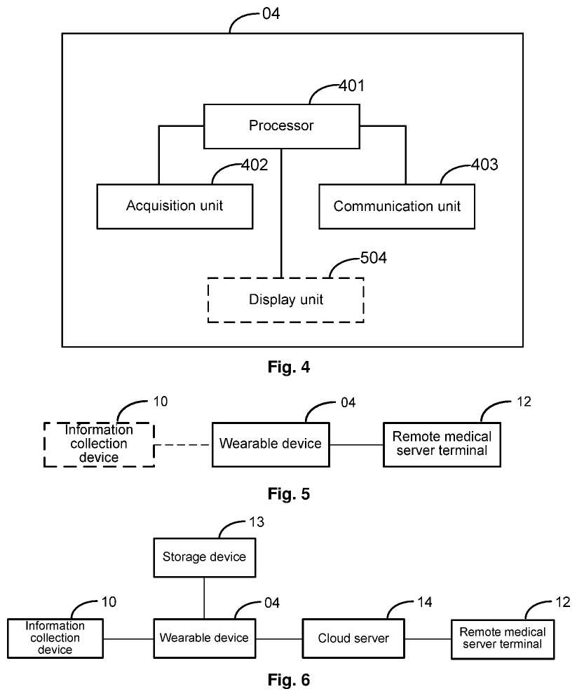

The embodiments of the present disclosure further provide a wearable device. As shown in FIG. 4, the wearable device 04 comprises:

an acquisition unit 402 configured to acquire pregnant woman health reference information;

a communication unit 403 configured to communicate with a remote medical server terminal; and

a processor 401, configured to:

control the acquisition unit to acquire the pregnant woman health reference information;

control the communication unit to transmit the pregnant woman health reference information and pregnant woman identity information in the wearable device to the remote medical server terminal; and

control the communication unit to receive pregnant woman health diagnosis information from the remote medical server terminal, wherein the pregnant woman health diagnosis information is determined according to the pregnant woman health reference information and the pregnant woman identity information.

In some embodiments, the wearable device 04 further comprises a display unit, configured to display at least one of the pregnant woman health reference information, the pregnant woman identity information, or the pregnant woman health diagnosis information.

In correspondence to the embodiment of the above health monitoring method, the wearable device according to the present disclosure may reduce the number of inspections in the medical institution, thereby reducing the time and labor costs, and improving the convenience and timeliness of monitoring the health of the pregnant woman.

The wearable device 04 may be used to implement the health monitoring method shown in FIGS. 1 to 3, and therefore, any explanation and description made above with respect to FIGS. 1 to 3 are equally applicable herein and will not be described again.

The apparatus embodiments described above are merely illustrative, wherein units described as separate components may or may not be physically separate, that is, the above units may be located in one place, or may be distributed over multiple network elements; and the above units may be combined into one unit, or may be further split into a plurality of sub-units.

With the above description, the apparatus according to the present embodiment may be implemented by means of software, or may be implemented by means of software plus necessary general hardware, or may of course also be implemented by means of hardware. Based on such understanding, the technical solutions according to the present disclosure or a part of the technical solutions contributing to the related art may be embodied in a form of software product. By taking a software implementation as an example, the apparatus, as a logical apparatus, is formed by reading corresponding computer program instructions in a non-volatile memory into a memory by a processor where the readable device is located.

The present disclosure further provides a computer readable storage medium having stored thereon executable instructions which, when executed by a processor, may implement the steps of the health monitoring method described in any of the above embodiments.

The machine-readable storage medium referred to herein may be any electronic, magnetic, optical, or other physical storage apparatus, which may contain or store information such as executable instructions, data, etc. For example, the machine-readable storage medium may be a Radom Access Memory (RAM), a volatile memory, a non-volatile memory, a flash memory, a storage driver (such as a hard disk driver), any type of storage disk (such as an optical disk, a dvd, etc.), or a similar storage medium, or a combination thereof.

The embodiments of the present disclosure further provide a health monitoring system. As shown in FIG. 5, the system comprises:

the wearable device 04 as shown in FIG. 4;

a remote medical server terminal 12 in communication with the communication unit in the wearable device, and configured to determine the pregnant woman health diagnosis information according to the pregnant woman health reference information and the pregnant woman identity information received from the wearable device, and transmit the pregnant woman health diagnosis information to the wearable device.

In some embodiments, the health monitoring system may further comprise an information collection device 10. The information collection device 10 is configured to collect the pregnant woman reference information and provide the collected pregnant woman reference information to the acquisition unit in the wearable device.

In some other embodiments, the health monitoring system does not comprise the information collection device 10. In this case, the acquisition unit in the wearable device 04 functions as the information collection device 10.

Specifically, the information collection device 10 may be a sensor for collecting the pregnant woman health reference information, comprising, for example, a fetal heart detection sensor, a temperature sensor, a pulse sensor, etc. The fetal heart detection sensor may be disposed on an abdominal surface of the pregnant woman to collect fetal heart information of the fetus in the pregnant woman. Specifically, the fetal heart detection sensor acquires a fetal heart signal by collecting a sound or vibration on the abdominal surface of the pregnant woman, and converts the fetal heart signal into a digital signal, which is transmitted to the wearable device as the fetal heart information.

The temperature sensor may also be disposed on the abdominal surface of the pregnant woman, to transmit the collected abdominal body surface temperature of the pregnant woman to the wearable device. The pulse sensor may be disposed on a wrist of the pregnant woman, to transmit the collected number of pulse beats of the pregnant woman to the wearable device. Alternatively, if the wearable device is a smart watch or a smart bracelet, the pulse sensor may be directly disposed on the smart watch or the smart bracelet, and the wearable device may directly acquire the collected number of pulse beats of the pregnant woman.

The above information collection device 10 may be implemented by using an existing sensor, which is not limited in the present disclosure.

It should be illustrated that, the wearable device may also have other functions, such as voice communication, video communication, WeChat communication, motion monitoring, music play, time display, calendar, etc., which is not limited in the present disclosure.

FIG. 6 is a block diagram of a health monitoring system according to another exemplary embodiment of the present disclosure. As shown in FIG. 6, the system comprises an information collection device 10, the wearable device 04 as shown in FIG. 4, a remote medical server terminal 12, a storage device 13, and a cloud server 14.

The wearable device 04 is further configured to transmit the pregnant woman health reference information and the pregnant woman identity information to the storage device 13 according to, for example, the received first voice instruction, and transmit the pregnant woman health reference information and the pregnant woman identity information to the cloud server 14 according to, for example, the received second voice instruction.

The storage device 13 is configured to store the received pregnant woman health reference information and pregnant woman identity information.

The cloud server 14 is configured to determine pregnant woman health information belonging to the same pregnant woman according to the received pregnant woman identity information, and establish a pregnant woman personal health file.

In the system, the cloud server 14 may further transmit the established pregnant woman personal health file to the remote medical server terminal 12.

Based on system architecture shown in FIG. 6, the remote medical server terminal 12 exchanges information with the wearable device 04 through the cloud server 14, and both the remote medical server terminal 12 and the wearable device 04 are connected to the cloud server 14 through a network. The wearable device 04 transmits the pregnant woman health reference information and the pregnant woman identity information to the cloud server 14, and after receiving the information, the cloud server 14 transmits the information to the remote medical server terminal 12. The remote medical server terminal 12 transmits the determined pregnant woman health diagnosis information to the cloud server 14, and the cloud server 14 feeds back the received information to the wearable device 04.

Further, in the system, as shown in FIG. 6, the wearable device 04 is further configured to store a pregnancy start time input by the pregnant woman, transmit the pregnancy start time to the cloud server 14, and receive and display a fetal development pattern map.

The cloud server 14 is further configured to calculate a current pregnancy time according to the pregnancy start time, construct a fetal development pattern map at the current pregnancy time, and transmit the fetal development pattern map to the wearable device 04.

In the above system, the information collection device may be connected to the wearable device in a wireless manner, such as Bluetooth or WIFI etc. The wearable device may be connected to the storage device and the cloud server through a wireless network, and the cloud server may be connected to the remote medical server terminal through a wireless network. The wireless network may be, for example, a 2G, 3G, 4G or 5G network etc.

The above health monitoring system integrates technologies such as the Internet of Things, information medicine and big data etc., and may monitor the health conditions of the pregnant woman in real time, which provides convenience for daily health monitoring of the pregnant woman.

It may be seen from the above technical solutions, the health monitoring method may be applied to the wearable device, and after the pregnant woman wears the wearable device, the pregnant woman may know the health conditions of herself and the fetus in her body in time according to the pregnant woman health reference information displayed by the wearable device, and may know professional health diagnosis information fed back by the remote medical server terminal in time, which may reduce a number of inspections in a medical institution, thereby reducing the time and labor costs, and improving the convenience and timeliness of monitoring the health of the pregnant woman.

Other embodiments of the present disclosure will be readily apparent to those skilled in the art after considering the specification and practicing the present disclosure. The present disclosure is intended to cover any variations, uses, or adaptations of the present disclosure, which comply with general principles of the present disclosure and comprise common general knowledge or conventional technical means in the art which are not disclosed in the present disclosure. The specification and embodiments are to be regarded as illustrative only, and the true scope and spirit of the present disclosure are defined by the claims.

I/We claim:

1. A health monitoring method performed by a wearable device, comprising:
acquiring pregnant woman health reference information;
transmitting the pregnant woman health reference information and pregnant woman identity information in the wearable device to a remote medical server terminal; and
receiving pregnant woman health diagnosis information from the remote medical server terminal, wherein the pregnant woman health diagnosis information is determined according to the pregnant woman health reference information and the pregnant woman identity information.

2. The health monitoring method according to claim 1, wherein the pregnant woman health reference information and the pregnant woman identity information in the wearable device are transmitted through a cloud server, and the pregnant woman health diagnosis information is received through the cloud server.

3. The health monitoring method according to claim 1, further comprising:
transmitting a pregnancy start time in the wearable device to a cloud server; and
receiving, from the cloud server, a fetal development pattern map at a current pregnancy time determined based on the pregnancy start time.

4. The health monitoring method according to claim 1, further comprising:
transmitting the pregnant woman health reference information and the pregnant woman identity information to a storage device for storage.

5. The health monitoring method according to claim 1, further comprising:
transmitting the pregnant woman health reference information and the pregnant woman identity information to a cloud server, so that the cloud server determines pregnant woman health reference information belonging to a same pregnant woman according to the pregnant woman identity information, and establishes a pregnant woman personal health file.

6. The health monitoring method according to claim 1, further comprising:
displaying at least one of the pregnant woman health reference information, the pregnant woman identity information, or the pregnant woman health diagnosis information.

7. A wearable device for health monitoring, comprising:
an acquisition unit configured to acquire pregnant woman health reference information;
a communication unit configured to communicate with a remote medical server terminal; and
a processor, configured to:
control the acquisition unit to acquire the pregnant woman health reference information;
control the communication unit to transmit the pregnant woman health reference information and pregnant woman identity information in the wearable device to the remote medical server terminal; and
control the communication unit to receive pregnant woman health diagnosis information from the remote medical server terminal, wherein the pregnant woman health diagnosis information is determined according to the pregnant woman health reference information and the pregnant woman identity information.

8. The wearable device according to claim 7, wherein the pregnant woman health reference information and the pregnant woman identity information in the wearable device are transmitted through a cloud server, and the pregnant woman health diagnosis information is received through the cloud server.

9. The wearable device according to claim 7, wherein the processor is further configured to:
control the communication unit to transmit a pregnancy start time in the wearable device to a cloud server; and
control the communication unit to receive, from the cloud server, a fetal development pattern map at a current pregnancy time determined based on the pregnancy start time.

10. The wearable device according to claim 7, wherein the processor is further configured to:
control the communication unit to transmit the pregnant woman health reference information and the pregnant woman identity information to a storage device for storage.

11. The wearable device according to claim 7, wherein the processor is further configured to:
control the communication unit to transmit the pregnant woman health reference information and the pregnant woman identity information to a cloud server, so that the cloud server determines pregnant woman health reference information belonging to a same pregnant woman according to the pregnant woman identity information, and establishes a pregnant woman personal health file.

12. The wearable device according to claim 7, further comprising a display unit, configured to display at least one of the pregnant woman health reference information, the pregnant woman identity information, or the pregnant woman health diagnosis information.

13. A computer readable storage medium, having stored thereon executable instructions which, when executed by a processor, implement the steps of the method according to claim 1.

14. A health monitoring system, comprising:
the wearable device according to claim 7; and
a remote medical server terminal in communication with the communication unit in the wearable device, and configured to determine the pregnant woman health diagnosis information according to the pregnant woman health reference information and the pregnant woman identity information received from the wearable device, and transmit the pregnant woman health diagnosis information to the wearable device.

15. The health monitoring system according to claim 14, further comprising:
an information collection device configured to collect the pregnant woman health reference information, and provide the collected pregnant woman health reference information to the acquisition unit in the wearable device.

16. The health monitoring system according to claim 14, further comprising:
a storage device, configured to store the pregnant woman health reference information and the pregnant woman identity information; and
a cloud server configured to determine pregnant woman health reference information belonging to a same pregnant woman according to the pregnant woman identity information, and establish a pregnant woman personal health file.

17. The health monitoring system according to claim 16, wherein the cloud server is further configured to receive a pregnancy start time in the wearable device, determine a current pregnancy time based on the pregnancy start time, construct a fetal development pattern map at the current pregnancy time, and transmit the fetal development pattern map to the wearable device.
